(19) United States
(12) Patent Application Publication
(10) Pub. No.: US 2024/0382693 A1
(43) Pub. Date: Nov. 21, 2024

(54) Injection System and Method

(71) Applicant: Onier Villarreal Alejandro, Odessa, FL (US)

(72) Inventor: Onier Villarreal Alejandro, Odessa, FL (US)

(21) Appl. No.: 18/320,976

(22) Filed: May 20, 2023

(51) Int. Cl. A61M 5/32

(52) U.S. Cl. CPC A61M 5/3205; A61M 5/3202; A61M 5/3287

(57) ABSTRACT

An injection system has a brace; a needle inside the brace in a retracted position; a mechanism operable to drive the needle from the retracted position to an extracted position; and a fluid dispenser fluidly connected to the needle. An adjustable fastener secures the brace to a body part. The injection system further includes an unmanned vehicle to carry and deploy the brace; a speaker; a microphone; a memory storing an application; and a processor which executes the application to cause the speaker and microphone to operate as a communication device and/or to cause the mechanism to extract the needle, cause the injection system to administer a dosage through the needle, and cause the mechanism to retract the needle. One or more sensors detect a position or pressure of the brace with respect to a body part. The needle may be an intravenous needle or an intraosseous needle.

### BACKGROUND

In emergency situations, including both in the civil and military contexts, often a patient is too far away from a medical facility while losing or having lost blood at dangerously high levels. In such situations, it is critical to promptly provide fluids and/or medications to the patient to ensure clinical stability. In an ideal situation, the patient would be provided with the right type of treatment intravenously or intraosseously in a clinical facility. In the field, there is a need for a device that can assist medical personnel, or even the individual patient alone, to quickly provide treatment to the patient in need of emergency medical assistance.

### SUMMARY

The features or aspects generally relate to an injection system and method, and more particularly relate to an injection system with a brace having a needle for the injection of fluids and medication to a patient.

This summary is provided to introduce a variety of concepts and/or aspects in a simplified form that is further disclosed in the detailed description, below. This summary is not intended to identify key or essential inventive concepts of the claimed subject matter, nor is it intended for determining the scope of the claimed subject matter.

An injection system has a brace with a needle inside the brace. The brace is placed on a patient and the needle is driven automatically or via manual operation. The brace has biosensors and a computing device capable of controlling the extraction and retraction of the needle. The computing device can also control fluids/medication and dosage provided to the patient through the needle, based on manual input and/or signals from the biosensors.

In one general aspect, an injection system comprises a brace; a needle inside the brace in a retracted position; a mechanism operable to drive the needle from the retracted position to an extracted position; and a fluid dispenser fluidly connected to the needle. The brace further comprises an adjustable fastener to secure the brace to a body part. Implementations may include features where the injection system further comprises an unmanned vehicle to carry and deploy the brace; a speaker; a microphone; a memory storing an application; and a processor which executes the application to cause the speaker and microphone to operate as a communication device. In some embodiments, the brace further comprises a beacon. In some embodiments, the brace further comprises one or more sensors to detect a position of the brace with respect to a body part. In some embodiments, the needle is an intraosseous needle. In some embodiments, the needle is an intravenous needle.

Implementations may include features where the injection system further comprises a memory storing an application; and a processor which executes the application to cause the mechanism to extract the needle, cause the injection system to administer a dosage through the needle, and cause the mechanism to retract the needle. In some embodiments, the brace further comprises one or more sensors to detect a position of the brace with respect to a body part; and wherein the processor which executes the application to cause the mechanism to extract the needle comprises the processor further executing the application to determine whether to cause the mechanism to extract the needle based on the position of the brace with respect to the body part.

In one general aspect, an injection system comprises a brace comprising one or more sensors to detect a position and an orientation of the brace with respect to a body part; a needle inside the brace in a retracted position; a mechanism to move the needle from the retracted position to an extracted position; and one or more fluid containers fluidly connected to the needle.

Implementations may include features where the brace further comprises an adjustable fastener to secure the brace to a body part. In some embodiments, the injection system of claim 10 further comprises an unmanned vehicle to carry and deploy the brace. In some embodiments, the brace further comprises a beacon. In some embodiments, the brace further comprises one or more sensors to detect a position of the brace with respect to a body part. In some embodiments, the needle is an intraosseous needle. In some embodiments, the needle is an intravenous needle.

In some embodiments, the injection system further comprises a memory storing an application; and a processor which executes the application to cause the mechanism to extract the needle, cause the injection system to administer a dosage through the needle, and cause the mechanism to retract the needle.

Implementations may include features where the processor which executes the application to cause the mechanism to extract the needle comprises the processor further executing the application to determine whether to cause the mechanism to extract the needle based on the position and the orientation of the brace with respect to the body part.

In one general aspect, a method comprises driving out a needle from a brace by a mechanism; and administering a dosage through the needle.

Implementations may include features where the method further comprises determining whether to cause the mechanism to drive out the needle based on the position of the brace with respect to a body part; and retracting the needle. In some embodiments, the method further comprises determining which fluid to administer and the dosage.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1-A illustrates a perspective view of an injection system, according to some embodiments.

FIG. 1-B illustrates a cross-sectional view of a brace fluidly connected to a fluid dispenser, according to some embodiments.

FIG. 1-C illustrates a perspective view of the injection system, according to some embodiments.

FIG. 1-D illustrates a perspective view of the injection system in wrapping form, according to some embodiments.

FIG. 2-A illustrates a top view of the brace with a computing system, according to some embodiments.

FIG. 2-B illustrates a block diagram of the computing system, according to some embodiments.

FIG. 3 illustrates a cross-section view of the injection system, according to some

embodiments

FIG. 4 illustrates a perspective view of a drone dropping a package containing the injection system, according to some embodiments.

FIG. 5 illustrates a flowchart of a process for using an injection system, according to some embodiments.

### DETAILED DESCRIPTION

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

As used herein, the terms “fluid” or “fluids” includes any fluid, medication, or injectable treatment.

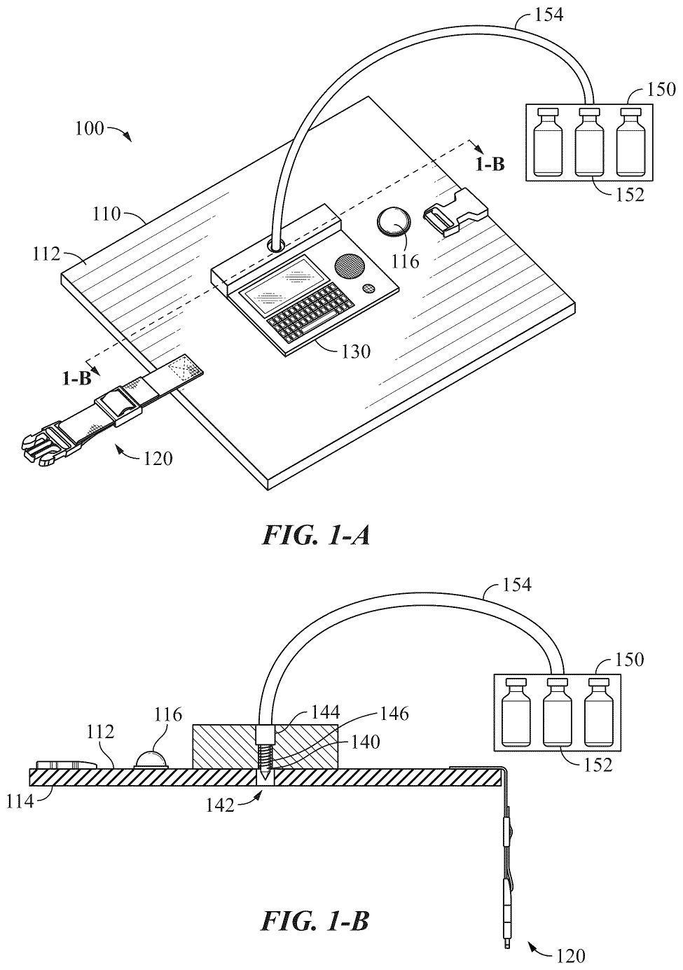

FIG. 1-A illustrates a perspective view of an injection system 100. FIG. 1-B illustrates a cross-sectional view of the brace 110 fluidly connected to the fluid dispenser 150. The injection system comprises a brace 110, a fluid dispenser 150, and a tube 154 from the fluid dispenser 150 to the brace 110.

The brace 110 comprises an outer surface 112, an inner surface 114, a beacon 116, an adjustable fastener 120, a computing system 130, and a needle 140. The tube 154 fluidly connects the fluid dispenser 150 to the needle 140 through a receiver 144. The needle 140 is shown in a retracted position in FIGS. 1-A and 1-B. The brace 110 is made of bendable rubber, foam, plastic, polymer, and/or the like. Both the outer surface 112 and the inner surface 114 are impermeable.

The beacon 116 is located on the outer surface 112 of the brace 110. A user may activate the beacon 116 through the computing system 130. In some embodiments, the computing system 130 automatically activates the beacon 116 when the computing system 130 is activated. The beacon may flash in frequencies outside the visible spectrum to allow specialized equipment to see the beacon 116, but not flash visually to individuals without the specialized equipment. This allows the beacon 116 to activate without visually alerting the location of the injection system 100 to enemy combatants.

The adjustable fastener 120 can be adjusted to fit bodies or body parts of various sizes. Generally, the brace 110 works best when positioned such that the needle 140 will provide intraosseous or intravenous access. In some embodiments, the needle 140 is an intraosseous needle. In some embodiments, the needle 140 is an intravenous needle. For intraosseous access, the brace 110 may be placed on the chest or on the front of the lower leg of a patient to access the humerus or the tibia, respectively. For intravenous access, the brace 110 may be placed on either arm of the patient. The brace 110 is placed on a body part with the inner surface 114 facing the body part. The inner surface 114 comprises biosensors 170. The biosensors 170 may be a variety of different sensors, including haptic sensors, galvanic skin response sensors, electrochemical biosensors, cameras, optical biosensors, electronic biosensors, piezoelectric biosensors, gravimetric biosensors, pyroelectric biosensors, magnetic biosensors, thermometric biosensors, and/or the like. The biosensors 170 capture a variety of signals, including heart rate, EKG, blood pressure, body temperature, pressure between the brace and a body part, and/or the like.

A needle driving mechanism 146 drives out the needle 140 through a needle opening 142 to an extracted position. The needle driving mechanism 146 also drives in the needle 140 through the needle opening 142 from the extracted position to the retracted position. The needle driving mechanism 146 is controlled by the computing system 130. In some embodiments, needle driving mechanism 146 is controlled by input or manual operation from a user that triggers the driving of the needle driving mechanism 146.

The fluid dispenser 150 comprises one or more fluid containers 152. The fluid dispenser 150 may be a backpack, a portable case, equipment in a drone, and/or the like. The fluid dispenser 150 is configured to fluidly connect at least one of the one or more fluid containers 152 to the needle 140. In some embodiments, the fluid dispenser 150, in response to instructions from the computing system 130, selects at least one of the one or more fluid containers 152 for injection. In some embodiments, the fluid dispenser 150, in response to input or manual operation from a user, selects at least one of the one or more fluid containers 152 for injection.

The one or more fluid containers 152 store different fluids that might be necessary for injection in an emergency. For example, the one or more fluid containers 152 store plasma, whole blood, platelets, colloids, crystalloids, tranexamic acid, antibiotics, and/or the like.

The computing system 130 is connected to and operates the needle driving mechanism 146. The computing system 130 determines whether to automatically drive the needle driving mechanism 146 based on the position and/or the pressure of the brace 110 with respect to a patient's body or body part. The computing system 130 determines the position and/or the pressure of the brace 110 with respect to the patient's body or body part based on the signals from the biosensors 170. In some embodiments, the computing system 130 visually and/or audibly indicates whether the brace 110 is in the correct position and/or has the correct pressure against the patient's body for extraction of the needle 140. In some embodiments, the computing system 130 receives input from a user that triggers the driving of the needle driving mechanism 146.

FIG. 1-C illustrates a perspective view of the injection system 100 from the side of the inner surface 114. The biosensors 170 are located throughout the inner surface 114. The needle 140 can be seen through the needle opening 142 located at the inner surface 114.

FIG. 1-D illustrates a perspective view of the injection system 100 in wrapping form. The wrapping form is the form in which the brace 110 is wrapped around a body part. For example, in operation, the brace 110 may be wrapped around the leg of a patient.

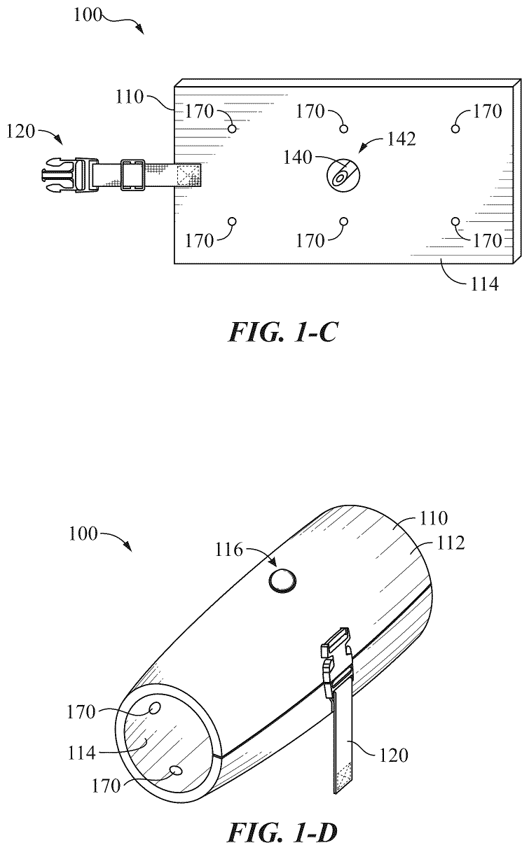

FIG. 2-A illustrates a top view of the brace 110 with the computing system 130. The computing system 130 determines which fluid or fluids from the one or more fluid containers 152 to request from the fluid dispenser 150 based on the signals from the biosensors 170. In some embodiments, the user manually selects and connects the one or more fluid containers 152 to the fluid dispenser 150. In some embodiments, the fluid containers are numbered, color coded, or otherwise identifiable, and the computing system 130 indicates to the patient or to another user which of the one or more fluid containers 152 to connect to the fluid dispenser 150. The fluid dispenser 150 receives the fluids from the one or more fluid containers 152, controlling the throughput of each fluid to the needle 140. FIG. 2-A shows the computing system 130 having a display 220, a keyboard 230, a speaker 240, and a microphone 250.

FIG. 2-B illustrates a block diagram of the computing system 130. Various features described within FIG. 2-B may generally complement the description of the other figures of the present disclosure. Moreover, a computing device such as the computing system 130 can be or be embedded in another device, e.g., a mobile telephone, a smartphone, a tablet, a laptop, desktop computer, electronic medical devices, a digital camera, a personal digital assistant (PDA), a mobile audio or video player, a game console, any global navigation system and/or global navigation satellite system transmitter/receiver/hardware/firmware/software (such as Global Positioning System (GPS), GLONASS, Galileo, BeiDou, QZSS, IRNSS/NavIC, or other global positioning, location, and/or navigation technology), a portable storage device (e.g., a universal serial bus (USB) flash drive), and/or the like.

The computing system 130 comprises a processor 210, a memory 214 storing an application 216, an input/output module or I/O module 218, the display 220, the keyboard 230, the speaker 240, and the microphone 250. The processor 210 is wired and/or wirelessly connected to the memory 214 and the I/O module 218. The I/O module 218 is wired and/or wirelessly connected to the biosensors 170, the display 220, the keyboard 230, the speaker 240, the microphone 250 and a network 260.

The processor 210 includes any suitable processor or processors implemented as a central processing unit (CPU), a graphics processing unit (GPU), an application-specific integrated circuit (ASIC), a field programmable gate array (FPGA), an artificial intelligence (AI) accelerator, any other type of processing unit, or a combination of different processing units, such as a CPU configured to operate in conjunction with a GPU. In general, processor 210 may be any technically feasible hardware unit capable of processing data and/or executing software applications such as the application 216. Further, in the context of this disclosure, the computing elements shown in the computing system 130 may correspond to a physical computing system (e.g., a local or networked computing device) or may be a virtual computing instance executing within a computing cloud.

Memory 214 includes a random-access memory (RAM) module, a read-only memory (ROM) module, a flash memory unit, a hard disk, magnetic disks, optical disks, magneto-optical disks, a mass storage device, a database, and/or any other type of memory unit or combination thereof. Processor 210 and I/O module 218 are configured to read instructions and/or data from and write instructions and/or data to memory 214. Memory 214 stores various software programs that can be executed by processor(s) 210 and application data associated with said software programs, including software that runs methods, steps, and processes such as the software, methods, steps, and/or processes described in this disclosure. The various software programs and the associated application data include the application 216.

The application 216 is configured to implement aspects described herein. The memory 214 stores various data accessible by the application 216. According to one or more aspects, the application 216 may include software elements corresponding to one or more of the various aspects described herein. For example, application 216 may be implemented in various aspects using any desired programming language, scripting language, or combination of programming languages and/or scripting languages (e.g., C, C++, C#, JAVA®, JAVASCRIPT®, PERL®, etc.). Furthermore, the code, instructions, data, functionality, and/or results from processing and/or execution of the application 216 can vary according to the person or use of the system, the data available, the device and/or user running the application, the role of the user and/or the device (for example, the user of the injection system 100 using the injection system 100 to obtain only his blood pressure), and/or the like.

The I/O module 218 provides the hardware, firmware, and/or software for the computing system 130 to interact with other components and devices, including the biosensors 170, the display 220, the keyboard 230, the speaker 240, the microphone 250, the network 260, other devices connected to the network 260, and/or the like. According to one or more aspects, the I/O module 218 can be connected to other devices, either wired or wirelessly, including input peripherals such as keyboards, microphones, cameras, and/or the like. The processor 210, through the I/O module 218, actuates, creates, generates, sends, and/or receives signals and/or data to and/or from the biosensors 170, the display 220, the keyboard 230, the speaker 240, the microphone 250, the network 260, other devices connected to the network 260, and/or the like. The I/O module 218 is configured to allow data to be exchanged between the computing system 130 and other devices attached to a network 260. According to one or more aspects, the I/O module 218 supports communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol, including WAN, LAN, Wi-Fi®, Bluetooth®, and/or the like.

The display 220 includes devices capable of providing output, such as screens, or both input and output, such as touchscreens. The display shows the status of the injection system 100, provides a menu for a user or the patient to operate the injection system 100, and shows patient biosignals such as heart rate, blood pressure, and the like. The user and/or the patient may use the display 220 or the keyboard 230 to provide input, commands, or otherwise operate the injection system 100. For example, the user and/or the patient, via the display 220 or the keyboard 230, may indicate to the application 216 and/or the computing system 130 that the patient and the brace 110 are ready for needle extraction.

The keyboard 230 includes any type of input device with buttons, including a touchscreen with a graphical interface of buttons. In some embodiments, the keyboard includes other interfaces, such as a pad to capture gestures that correspond to movements of a cursor or pointer on the display 220.

The speaker 240 includes any type of sound emitting device, including wired or wireless speakers. For example, in some embodiments, the user or the patient is wearing a headset or headphones that receive sound signals wirelessly from the computing device 130 through the network 260. Likewise, the microphone 250 includes any type of sound capturing device, including wired or wireless microphones. For example, in some embodiments, the user or the patient is wearing a headset or headphones with microphone that send sound signals wirelessly to the computing device 130 through the network 260.

The network 260 includes any technically feasible type of communications network that allows data to be exchanged between the computing system 130 and external entities or devices. For example, the network 260 may include a wide area network (WAN), a local area network (LAN), a wireless (Wi-Fi®) network, Bluetooth®, and/or the Internet, among others (including any communication channel, medium, and/or network accessible by the I/O module 218). According to one or more aspects, the I/O module 218 is not directly connected to one or more elements, parts, components, devices and/or networks. According to one or more aspects, the I/O module 218 connects wired and/or wirelessly to the to one or more elements, parts, components, devices and/or networks through the network 260.

Relying on the network 260, the display 220, the keyboard 230, the speaker 240, and/or the microphone 250 cooperate to work as a communication device and/or a Combat Survivor Evader Locator (CSEL) system via execution of the application 216 by the processor 210.

Also, any connection may be associated with a computer-readable medium or media. For example, if the application 216 and/or data associated with or operated by the application 216 is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Storage of any kind data of the application 125 and/or data associated with or operated by the application 216 such as hard discs, disks, RAM, ROM, flash memory, cloud storage, and/or the like (including the memory 214) is also included in the definition of medium. “Disk” and “disc,” as used herein, include compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

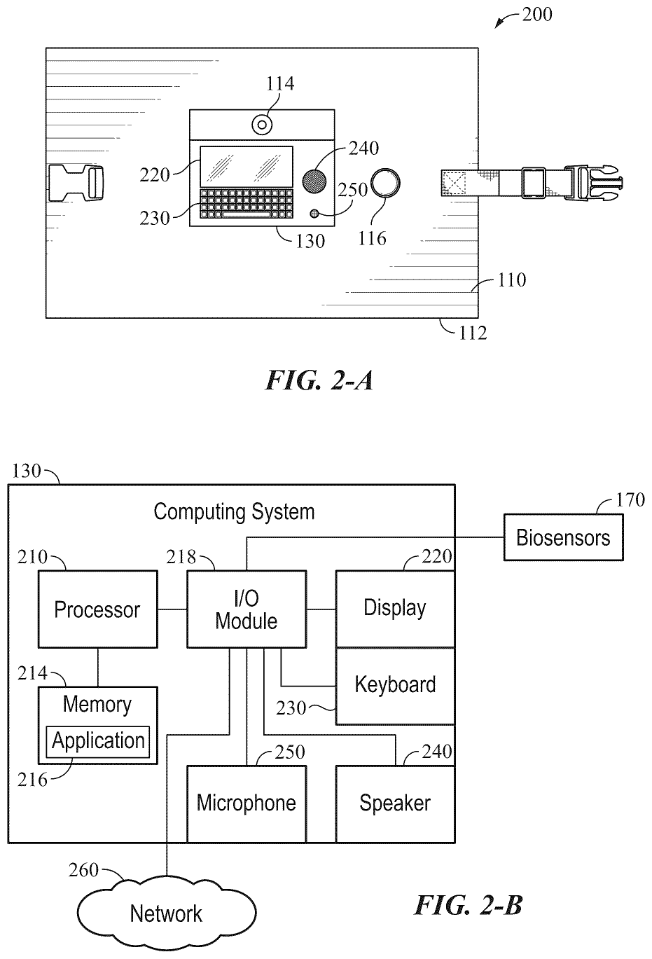

FIG. 3 illustrates a cross-section view of the injection system 100 without the fluid dispenser 150 and without the tube 154. Any of the one or more fluid containers 152 can be selected for dosage. A selected container 310 has a cap 320 that fits the receiver 144.

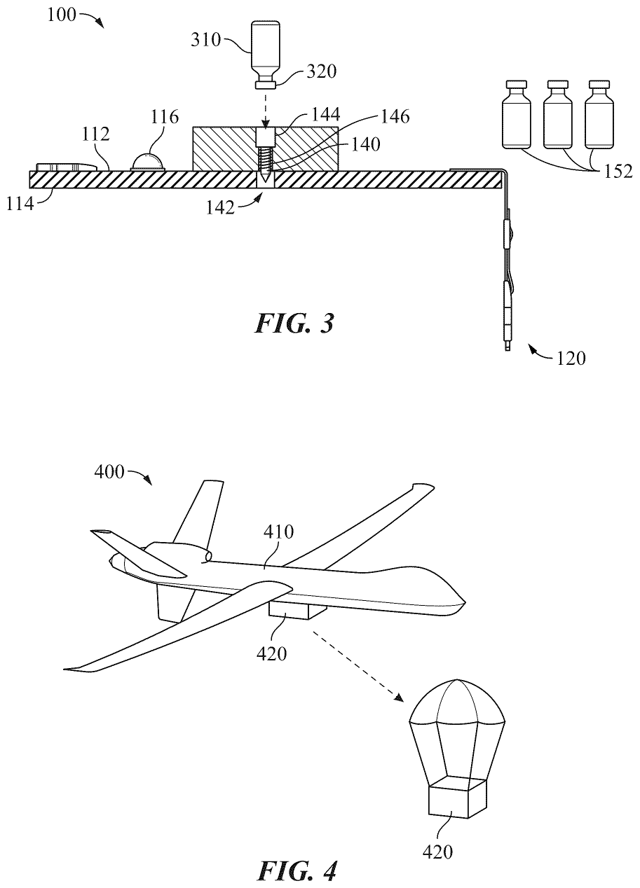

FIG. 4 illustrates a perspective view of a drone or UAV (Unmanned Aerial Vehicle) 410 dropping a package 420 containing the injection system 100. The drone 410 can augment the medical features and/or the communication capabilities of the injection system 100. In some embodiments, the drone 410 has hardware that adds to or complements the communication capabilities of the computing device 130. In some embodiments, the drone 410 relays the communications to and from the computing device 130.

To enable the reader to obtain a clear understanding of the technological concepts described herein, the following process describes specific steps performed in a specific order. However, one or more of the steps of a particular process may be rearranged and/or omitted while remaining within the contemplated scope of the technology disclosed herein. One or more processes and/or steps thereof, may be combined, recombined, rearranged, omitted, or executed in parallel to create different process flows that are within the contemplated scope of the technology disclosed herein. While the process below may omit or briefly summarize some of the details of the technologies disclosed herein for clarity, the details described in the paragraphs above may be combined with the process steps described below to get a more complete and comprehensive understanding of these processes and the technologies disclosed herein.

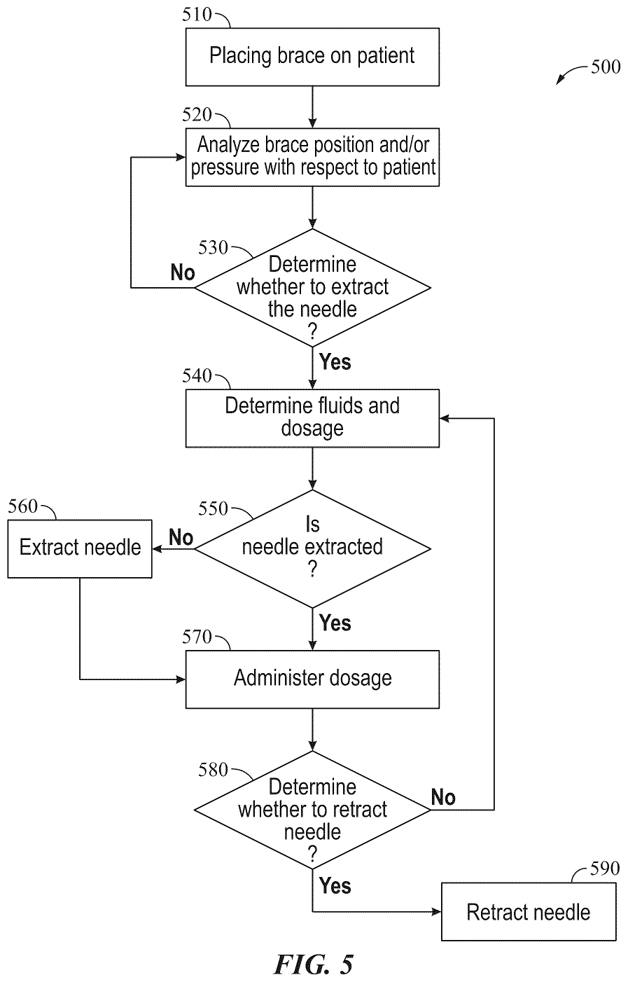

FIG. 5 illustrates a flowchart of a process 500 for using an injection system such as the injection system 100. The process starts at step 510, where the brace 110 is placed on the patient. In operation, the brace 110 is secured to the patient, for example, at the chest or at the lower leg. Next, in step 520, the position and/or the pressure of the brace 110 with respect to the patient or the patient's body part are analyzed. This analysis can be done by the computing system 130 based on signals from the biosensors 170. The biosensors 170 send signals that are processed by the computing system 130 to determine the position of the brace 110 with respect to the patient's body, the pressure between the patient's body and the brace 110, blood pressure, heart rate, and/or the like. In some embodiments, the patient or a user may input to the computing system 130 the position of the brace 110 with respect to the patient's body and/or input a confirmation that the brace 110 has been properly positioned and secured to the patient.

Process 500 continues with step 530, where it is determined whether to extract the needle based on whether the brace 110 is correctly positioned, whether the fluids from the fluid dispenser 150 and/or the one or more fluid containers 152 are accessible to the needle 140, and/or whether the appropriate fluid is fluidly connected to the needle 140. The determination of whether to extract the needle may be made manually by the patient or a user or automatically by the computing system 130 based on the signals from the biosensors 170. If it is determined that the needle should not be extracted, the process goes back to step 520 to analyze the position of the brace 110. If it is determined that the needle 140 should be extracted, the process moves on to step 540.

In step 540, the fluids to be administered and the dosage are determined. The determinations of which fluids to administer and what dosage to administer are done manually by the patient or a user and/or done automatically by the computing system 130 based on the signals from the biosensors 170.

In step 550, the computing system 130 determines whether the needle 140 is already extracted. Note that at step 550 it has already been determined that the needle should be extracted at step 530 and the fluids and dosage have already been determined at step 540. If the needle is already extracted, the process 500 moves on to step 570. If the needle is not extracted at step 550, the process 500 moves to step 560 to extract the needle.

In step 560, the needle 140 is extracted, driven into the patient by the needle driving mechanism 146. In step 570, and the dosage of fluids is administered to the patient. In some embodiments, the needle is extracted when both the position of the brace 110 is detected to be appropriate or user input approves the position of the brace 110, and the fluids and dosage are determined. In some embodiments, the determination of fluids includes the computing system 130 communicating to the fluid dispenser 150 which fluids to provide to the needle 140. In some embodiments, the determination of fluids includes choosing the selected container 310 from the one or more fluid containers 152 and fluidly connecting the selected container 310 to the receiver 144.

Next, in step 580, the computing system 130 determines whether to retract the needle based on fluids previously administered, the signals from the biosensors 170, and/or user or patient input. If it is determined that the needle is not to be retracted, the process 500 moves to step 540 to determine whether changes to the fluids and/or dosages are required. If it is determined that the needle is to be retracted, the process 500 moves to step 590 to cause the needle driving mechanism 146 to retract the needle.

What is claimed is:

1. An injection system comprising:
a brace;
a needle inside the brace in a retracted position;
a mechanism operable to drive the needle from the retracted position to an extracted position; and
a fluid dispenser fluidly connected to the needle.

2. The injection system of claim 1, the brace further comprising:
an adjustable fastener to secure the brace to a body part.

3. The injection system of claim 1 further comprising:
an unmanned vehicle to carry and deploy the brace;
a speaker;
a microphone;
a memory storing an application; and
a processor which executes the application to:
cause the speaker and microphone to operate as a communication device.

4. The injection system according to claim 1, wherein the brace further comprises a beacon.

5. The injection system according to claim 1, wherein the brace further comprises:
one or more sensors to detect a position of the brace with respect to a body part

6. The injection system according to claim 1, wherein the needle is an intraosseous needle.

7. The injection system according to claim 1, wherein the needle is an intravenous needle.

8. The injection system of claim 1 further comprising:
a memory storing an application; and
a processor which executes the application to:
cause the mechanism to extract the needle,
cause the injection system to administer a dosage through the needle, and
cause the mechanism to retract the needle.

9. The injection system according to claim 8, wherein the brace further comprises:
one or more sensors to detect a position of the brace with respect to a body part; and
wherein the processor which executes the application to cause the mechanism to extract the needle comprises:
the processor further executing the application to:
determine whether to cause the mechanism to extract the needle based on the position of the brace with respect to the body part.

10. An injection system comprising:
a brace comprising one or more sensors to detect a position and an orientation of the brace with respect to a body part;
a needle inside the brace in a retracted position;
a mechanism to move the needle from the retracted position to an extracted position; and
one or more fluid containers fluidly connected to the needle.

11. The injection system of claim 10, the brace further comprising:
an adjustable fastener to secure the brace to a body part.

12. The injection system of claim 10 further comprising:
an unmanned vehicle to carry and deploy the brace.

13. The injection system according to claim 10, wherein the brace further comprises a beacon.

14. The injection system according to claim 10, wherein the needle is an intraosseous needle.

15. The injection system according to claim 10, wherein the needle is an intravenous needle.

16. The injection system of claim 10 further comprising:
a memory storing an application; and
a processor which executes the application to:
cause the mechanism to extract the needle,
cause the injection system to administer a dosage through the needle, and
cause the mechanism to retract the needle.

17. The injection system according to claim 16, wherein the processor which executes the application to cause the mechanism to extract the needle comprises:
the processor further executing the application to:
determine whether to cause the mechanism to extract the needle based on the position and the orientation of the brace with respect to the body part.

18. A method comprising:
driving out a needle from a brace by a mechanism; and
administering a dosage through the needle.

19. The method of claim 18 further comprising:
determining whether to cause the mechanism to drive out the needle based on the position of the brace with respect to a body part; and
retracting the needle.

20. The method of claim 19 further comprising:
determining which fluid to administer and the dosage.
